(19) United States
(12) Patent Application Publication
(10) Pub. No.: US 2026/0198910 A1
(43) Pub. Date: Jul. 16, 2026

(54) Throat Specimen Collection Devices with a Tongue Depressor for Positioning the Collection Receptacle

(71) Applicant: Abbott Diagnostics Scarborough Inc., Scarborough, ME (US)

(72) Inventors: Victor Shapiro, Scarborough, ME (US); Bryan Johnson, San Diego, CA (US); Bryce Gibson, Scarborough, ME (US); Gourab Saha, San Diego, CA (US); Jocelyn Roncal, Scarborough, ME (US)

(21) Appl. No.: 19/133,477

(22) PCT Filed: Dec. 1, 2023
(86) PCT No.: PCT/US2023/082193

§ 371 Date: May 28, 2025

Related U.S. Application Data
(60) Provisional application No. 63/429,365, filed on Dec. 1, 2022.

(51) Int. Cl. A61B 13/00; A61B 1/06; A61B 10/00

(52) U.S. Cl. CPC A61B 13/00; A61B 1/06; A61B 10/0051

(57) ABSTRACT

Provided are throat specimen collection devices and methods of using them. In certain embodiments, a throat specimen collection device comprises: i) a tongue depressor configured to restrain a subject's tongue, and ii) a collection receptacle comprising, an elongated member and a collection member, wherein, when the tongue depressor is positioned to depress a subject's tongue and the collection receptacle is in a collection position, the collection member touches the throat of the subject and collects the throat specimen from the subject. Methods of collecting throat specimens are also provided, the methods comprising, employing a throat specimen collection device to a subject's mouth and collecting a throat specimen.

### CROSS-REFERENCE TO RELATED APPLICATIONS

This application claims the benefit of U.S. Provisional Patent Application No. 63/429,365, filed Dec. 1, 2022, which application is incorporated herein by reference in its entirety.

### INTRODUCTION

Collecting throat specimens typically involves a health-care professional to introduce a throat specimen collection apparatus into a subject's mouth. Throat specimen collection apparatuses are generally elongated structures with a throat specimen collection receptacle at the end. A health-care professional introduces the throat specimen collection apparatus into a subject's open mouth and touches the throat with the collection receptacle.

These devices and methods are often unpleasant for the subjects. For example, certain patients, particularly, children exhibit anxiety with the collection devices and procedures. Certain patients may experience unpleasant gagging during the collection. Therefore, collecting throat specimens from subjects is often challenging and unpleasant for both the subjects as well as the health-care professionals.

### SUMMARY

The disclosure provides throat specimen collection devices and methods of collecting throat specimens using such devices. The throat specimen collection devices disclosed herein address the problems discussed above with prior devices and collection methods.

The throat specimen collection devices and methods disclosed herein can be used to avoid some of the unpleasant experience in throat specimen collection. For example, the throat specimen collection devices and methods disclosed herein, when used in children may reduce the child's anxiety during throat specimen collection. Also, the throat specimen collection devices and methods disclosed herein may avoid unpleasant gagging during collection in certain subjects.

In certain embodiments, a throat specimen collection device comprises:
i) a tongue depressor, and
ii) a collection receptacle comprising, an elongated member and a collection member,
wherein, when the tongue depressor is positioned to depress a subject's tongue and the collection receptacle is in a collection position, the collection member touches the throat of the subject and collects the throat specimen from the subject.

The collection receptacle may comprise an elongated member having at one end a collection member. The collection member may be a cotton swab or a brush head, such as a nubby silicon brush head. The collection member may also be a rayon swab, polyester swab, foam swab, or flocked swab.

A tongue depressor can be configured to restrain a subject's tongue. For example, a tongue depressor can be longitudinal and flat, cylindrical, or popsicle shaped to fit in a subject's mouth, particularly, over the subject's tongue. The tongue depressor can be made of any suitable material, such as wood, plastic, rubber, cardboard paper, or silicon. The tongue depressor can also be flavored.

When a tongue depressor is positioned to restrain a subject's tongue, a collection receptacle can be in a collection position where a collection member touches the subject's throat. Alternatively, when a tongue depressor is positioned to restrain a subject's tongue, a collection receptacle can be in an introduction position where a collection member does not touch the subject's throat. The collection receptacle can then be moved from the introduction position to the collection position. For example, the collection receptacle in the introduction position can be moved through the tongue depressor into the collection position.

In some cases, the collection receptacle in the introduction position can be slid over the tongue depressor into the collection position. Such sliding over can be via a slidable engagement between the collection receptacle holder and the tongue depressor. In even further embodiments, when in operation, the collection receptacle is physically separate from the tongue depressor.

In some cases, a throat specimen collection device comprises an illuminator element. The illuminator element can be automated or operated by a user. The illuminator element is positioned in a collection device such that, when in use, the illuminator element illuminates the inside of a subject's mouth thereby making the subject's mouth more visible and facilitating a better control in specimen collection, for example, better control of the location inside the subject's mouth from where the specimen is collected.

Also provided are methods of collecting throat specimens using the throat specimen collection devices. Such methods comprise:
i) positioning the tongue depressor of the throat specimen collection device to restrain a subject's tongue, and
ii) collecting a throat specimen of the subject when the collection receptacle is in the collection position.

In certain cases, the method comprises moving the collection receptacle from an introduction position to a collection position. In further cases, the method comprises illuminating the subject's mouth using an illuminator element.

### BRIEF DESCRIPTION OF THE DRAWINGS

FIG. 1 shows an embodiment of a throat specimen collection device.

FIG. 2 shows an embodiment of a throat specimen collection device.

FIG. 3 shows another embodiment of a throat specimen collection device.

FIG. 4 shows a further embodiment of a throat specimen collection device.

FIG. 5 shows an example of a nubby silicon brush head that can be used as a collection member.

FIG. 6 shows a further embodiment of a throat specimen collection device.

FIG. 7 shows the throat specimen collection device of FIG. 6 in operation.

FIG. 8 shows an embodiment of a throat specimen collection device comprising an illuminator element.

FIG. 9 shows certain components inside an exemplary throat specimen collection device of FIG. 8.

FIG. 10 shows an exemplary throat specimen collection device of FIGS. 8 and 9 in operation.

FIG. 11 shows an embodiment of a throat specimen collection device.

FIG. 12 shows an exemplary throat specimen collection device of FIG. 11 in operation.

### DETAILED DESCRIPTION

Provided are throat specimen collection devices and methods of collecting throat specimens using the devices.

In certain embodiments, a throat specimen collection device comprises:
i) a tongue depressor, and
ii) a collection receptacle comprising an elongated member and a collection member,
wherein, when the tongue depressor is positioned to depress a subject's tongue and the collection receptacle is in a collection position, the collection member touches the throat of the subject and collects the throat specimen from the subject.

Also provided are methods of collecting throat specimens using the throat specimen collection devices disclosed herein. Certain such methods comprise:
i) positioning a tongue depressor of a throat specimen collection device to restrain a subject's tongue, and
ii) collecting a throat specimen of the subject when the collection receptacle is in the collection position.

Before the present disclosure is described in greater detail, it is to be understood that this disclosure is not limited to particular embodiments described, as such may vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a device” includes a plurality of such devices. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

### Definitions

As used herein, the term “a throat specimen” refers to a biological sample obtained from the back of a subject's mouth from the area at the top part of the tubes that go down into the subject's stomach and lungs. Throat specimens typically comprise microbes from the throat of a subject and may also contain the subject's cells, mucus, saliva, and other secretions. The term specimen may be used interchangeably with “throat sample” and retain the same meaning.

A “swab” is an absorbent pad or piece of material used for collecting specimens.

“A tongue depressor” refers to a part of the throat specimen collection device that is positioned in a subject's mouth to restrain the subject's tongue.

A “collection member” as used herein refers to a part of the collection receptacle that is configured to obtain a specimen from a subject's throat. The collection receptacle comprises an elongated member having at one end a collection member which can be a swab, for obtaining a specimen from a subject's throat.

In certain embodiments, the throat specimen collection devices comprise a collection receptacle holder. A collection receptacle can be physically fitted into a collection receptacle holder. For example, a collection receptacle can be available as consumables for use with the throat specimen collection devices disclosed herein. A collection receptacle can also be physically integral to a collection receptacle holder.

The term “restrain a subject's tongue” indicates that the tongue depressor covers the subject's tongue and holds it in place. The subject may still be able to move his/her tongue with some force, but without any intentional tongue movement by the subject, a tongue depressor can be used to hold a subject's tongue in position with moderate efforts by a user, for example, the subject or a health-care professional who is collecting the throat specimen.

### Throat Specimen Collection Devices

As described above, in certain embodiments, the throat specimen collection device comprises:
i) a tongue depressor, and
ii) a collection receptacle comprising, an elongated member and a collection member,
wherein, when the tongue depressor is positioned to depress a subject's tongue and the collection receptacle is in a collection position, the collection member touches the throat of the subject and collects the throat specimen from the subject.

The collection receptacle can be a physically integrated element of the throat specimen collection device. Alternatively, the collection receptacle can be a physically separate unit but can be physically fitted into the tongue depressor to constitute a throat specimen collection device.

In some cases, when a tongue depressor is positioned to restrain a subject's tongue, the collection receptacle is in the collection position. In the collection position, the collection receptacle is positioned such that the collection member touches the throat of the subject and collects a throat specimen from the subject.

In some cases, the collection receptacle is movable between an introduction position and the collection position. An introduction position of the collection receptacle is the position in which the collection member does not touch the throat of the subject, for example, when the collection receptacle is introduced into a tongue depressor. A collection receptacle in the introduction position can be moved into the collection position, where the collection member touches the throat of a subject. In some cases, the collection receptacle in the introduction position is slidable through the tongue depressor into the collection position.

In some cases, the collection receptacle in the introduction position is slidable into the collection position over the tongue depressor via a slidable engagement between the tongue depressor and a collection receptacle holder.

In some cases, when a tongue depressor is positioned to restrain a subject's tongue, the collection receptacle is in an introduction position, where the collection member does not touch the throat of a subject. The collection receptacle in an introduction position can be moved into the collection position, where the collection member touches the throat of a subject and collects the throat specimen from the subject.

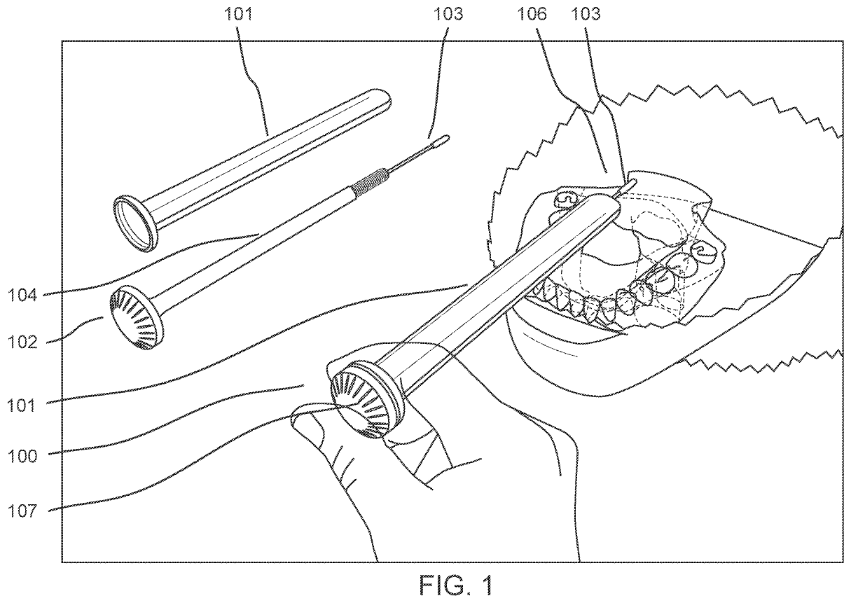

A non-limiting example of a throat specimen collection device is described in FIG. 1. In this embodiment, the throat specimen collection device 100 is placed into a subject's mouth to restrain the subject's tongue. Particularly, a tongue depressor 101 can be placed over the subject's tongue to restrain the subject's tongue.

As shown in FIG. 1, the collection receptacle 102 is in the collection position 106 where the collection member 103 touches the throat of the subject. Before the collection member touches the throat of the subject, a user, who can be the subject or another person, for example a health-care professional, can introduce the collection receptacle 102 into the tongue depressor 101 in an introduction position, where the collection member does not touch the subject's throat. A user can then move the collection receptacle from the introduction position into the collection position 106 by pushing the collection receptacle 102 through the tongue depressor 101 thereby pushing the collection member 103 towards the subject's throat and into the collection position 106. In the collection position 106, the collection member 103 touches the throat of a subject and collects the throat specimen from the subject. The collection receptacle can also comprise the knob 107, which can be used to grip the collection receptacle 102, push it through the tongue depressor 101, or turn or spin the collection member 103 when in the collection position 106 thereby facilitating collection of a throat specimen.

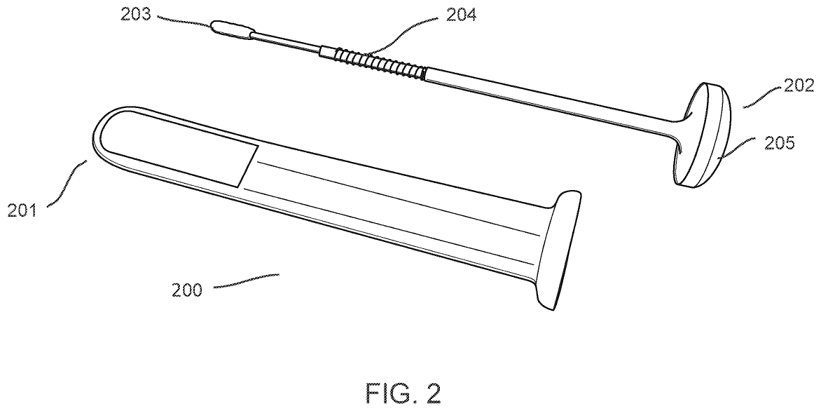

In some cases, the collection receptacle is physically separate from the tongue depressor, for example, as shown in FIGS. 1 and 2. In certain cases, when the tongue depressor is positioned in a subject's mouth to restrain the subject's tongue, the collection receptacle can be introduced into the tongue depressor at an introduction position, where the collection member does not touch the throat of a subject. The collection receptacle in the introduction position can then be moved into the collection position, where the collection member touches the throat of a subject and collects the throat specimen from the subject.

For example, as shown in FIG. 2, the collection receptacle 202, which is separate from the tongue depressor 201, can be introduced into the introduction position after the tongue depressor is positioned to restrain a subject's tongue. A user, who can be the subject or another person, for example a health-care professional, can move the collection receptacle from the introduction position into the collection position by pushing on the knob 205 which in turn pushes the collection member 203 towards the subject's throat and into the collection position. In the collection position, the collection member 203 touches the throat of a subject and collects the throat specimen from the subject. After the collection of a throat specimen, the spring 204 in the collection receptacle 202 can facilitate retraction of the collection receptacle 203 from the collection position.

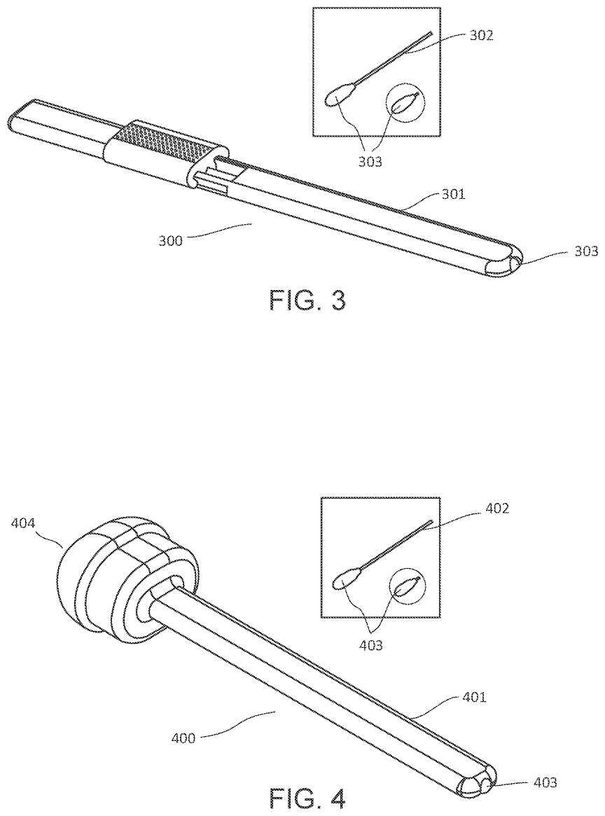

Additional non-limiting examples of embodiments in which the tongue depressors and the collection receptacles are physically separate are provided in FIGS. 3 and 4. In FIGS. 3 and 4, the collection receptacles 302 and 402 can be physically fitted into the tongue depressors 301 and 401 to constitute the throat specimen collection devices 300 and 400. In these embodiments, the tongue depressor 301 or 401 can be used to restrain a subject's tongue. The collection receptacle 302 or 402, which is physically separate from the tongue depressor 301 or 401, can be fitted into the introduction position where the collection member 303 or 403 does not touch the throat of the subject. A user, who can be the subject or another person, such as a health-care professional, can then move the collection receptacle from the introduction position into the collection position, where the collection member 303 or 403 touches the throat of a subject and collects the throat specimen from the subject.

The size of a tongue depressor can be varied depending on the size of a subject's mouth. For example, a shorter tongue depressor can be used for a child whereas a longer tongue depressor can be used for an adult.

In some cases, when a tongue depressor is used to restrain a subject's tongue, an introduction position of the collection receptacle can be towards the subject's lips. Thus, the collection receptacle can be introduced into a subject's mouth near the subject's lips and the collection receptacle can then be moved into the subject's mouth such that the collection member touches the subject's throat.

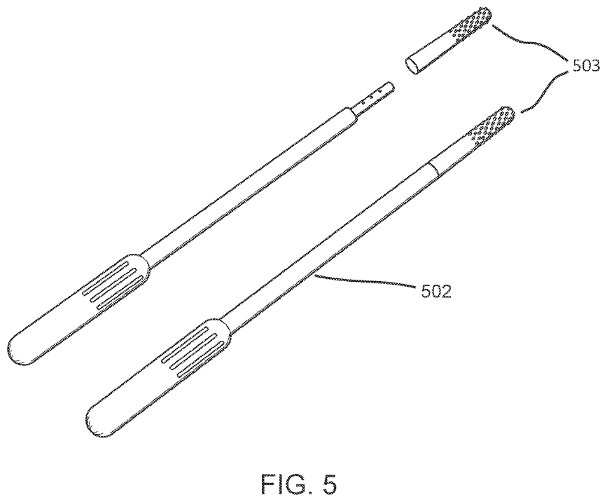

In some cases, a throat specimen collection receptacle is a longitudinal member 502 having at one end the collection member 503 for collecting the throat specimen. A collection member for collecting the throat specimen can be a cotton swab or a brush head, such as a nubby silicon brush head 503 as shown in FIG. 5. The collection member may also be a rayon swab, polyester swab, foam swab, or flocked swab.

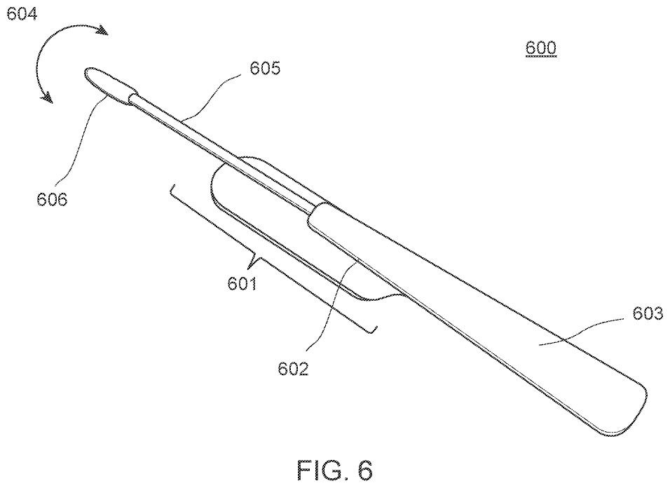

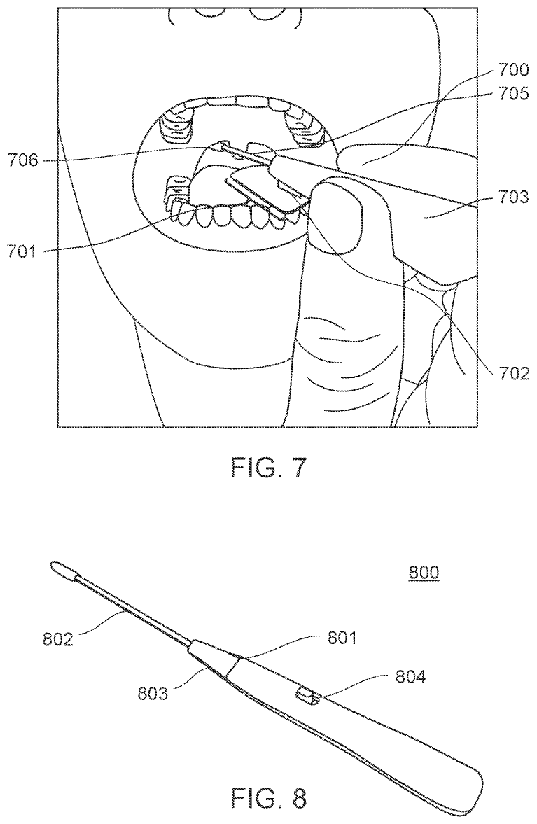

In some cases, a throat specimen collection device comprises a tongue depressor in a slidable engagement with a collection receptacle holder. Exemplary embodiments of such throat specimen collection devices are provided in FIGS. 6 and 7. In the throat specimen collection device 600 depicted in FIG. 6, a longitudinal and flat tongue depressor 601 is used to restrain a subject's tongue. The tongue depressor 601 has a slidable engagement 602 with the collection receptacle holder 603. The slidable engagement 602 can comprise an elongated body, for example, an elongated cylindrical body, which engages with the collection receptacle holder via a corresponding elongated slot in collection receptacle holder, which fits over the elongated body such that the collection receptacle holder slides over the elongated body of the tongue depressor.

In addition to the slidable engagement, the tongue depressor holder may also have a rotatable engagement with the tongue depressor. Using such rotatable engagement, a user can rotate the collection receptacle holder along the directions indicated by the two headed arrow 604. Such a rotatable movement facilitates scraping of a subject's throat using the collection member 606 thereby collecting a throat specimen of the subject.

In some cases, a collection receptacle is physically fitted on the collection receptacle holder 603. For example, the collection receptacle shown as 302 or 402 in FIGS. 3 and 4 could be physically fitted on the collection receptacle holder 603. Alternatively, the collection receptacle 605 can be physically integral with the collection receptacle holder 603.

FIG. 7 shows an exemplary embodiment of the throat specimen collection device of FIG. 6 in operation. When the device 700 of FIG. 7 is in operation, a user restricts a subject's tongue using the tongue depressor 701. The collection receptacle 705 is in introduction position (not shown), where the collection member 706 does not touch the subject's throat. The user can move the collection receptacle from the introduction position into the collection position where the collection member 706 touches the subject's throat to collect a throat specimen. Such movement can be achieved by sliding the collection receptacle holder 703 over the tongue depressor using the slidable engagement 702 between the two so that the collection receptacle slides over the tongue depressor and the collection member touches the subject's throat and collects a throat specimen.

When the tongue depressor has a rotatable engagement with the collection receptacle holder, a user can use such rotatable engagement to rotate the collection receptacle along the directions indicated by the two headed arrow 604. Such rotatable movement facilitates scraping of a subject's throat using the collection member 606 thereby collecting a throat specimen of the subject.

In some cases, a throat specimen collection device comprises an illuminator element. FIG. 8 shows one such embodiment of a throat specimen collection device. In this embodiment the throat specimen collection device 800 comprises the illuminator element 801, such as an LED, which, when in use, illuminates the inside of the subject's mouth. The illuminator element can be turned on and off using the switch 804. The collection receptacle 802 can either be physically fitted on the collection receptacle holder 803 or be physically integral with the collection receptacle holder 803.

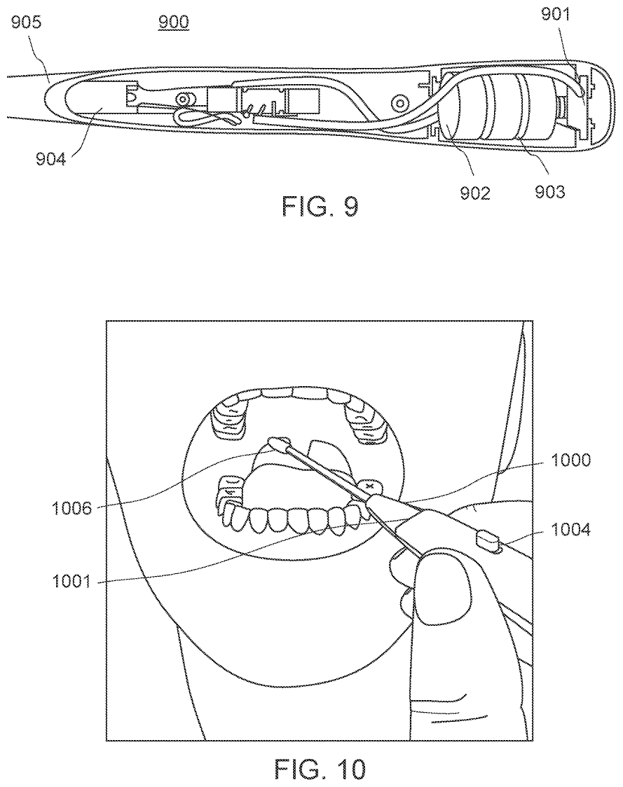

FIG. 9 shows certain components inside an exemplary throat specimen collection device of FIG. 8. The throat specimen collection device 900 comprises the illuminator element 904, which is powered through the power source, such as batteries, 903. The batteries are connected to the illuminator element 904 through terminals 901 and 902.

FIG. 10 shows an exemplary throat specimen collection device of FIGS. 8 and 9 in operation. A user turns on the illuminator element 1001 on the throat specimen collection device 1000 using the switch 1004. When the throat specimen collection device 1000 is placed into a subject's mouth, the illuminator element 1001 illuminates the inside of the subject's mouth. The user can then touch the subject's throat with the collection member 1006 to collect the subject's throat specimen.

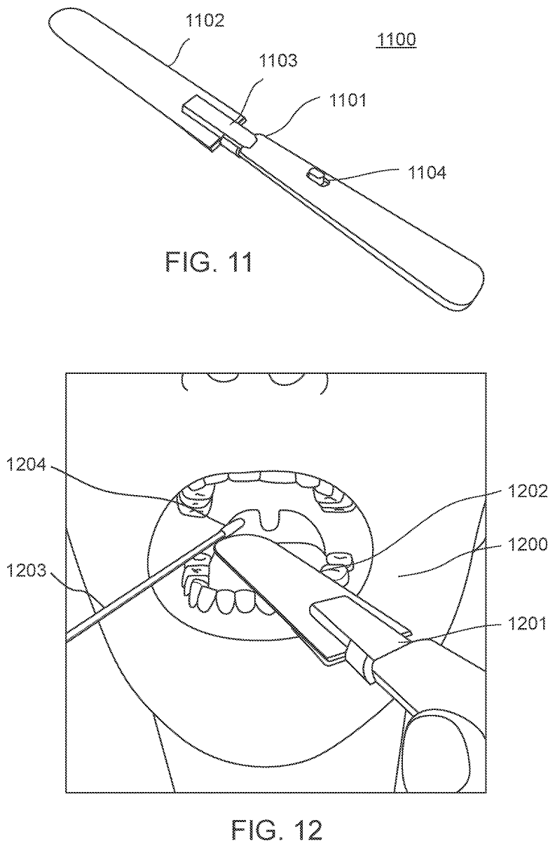

FIG. 11 shows another embodiment of the throat specimen collection device. In this embodiment, the throat specimen collection device of FIG. 10 further comprises a tongue depressor. In FIG. 11, the throat specimen collection device 1100 comprises the illuminator element 1101, which a user can turn on using the switch 1104. The throat specimen collection device further comprises a tongue depressor mounting element 1103 onto which the tongue depressor 1102 can be physically fitted. Alternatively, the tongue depressor 1102 can be physically integral to the throat specimen collection device.

FIG. 12 shows an exemplary throat specimen collection device of FIG. 11 in operation. When the throat specimen collection device 1200 is placed into a subject's mouth, the illuminator element 1201 illuminates the inside of the subject's mouth. A user can turn on the illuminator element 1201 and restrict a subject's tongue with the tongue depressor 1202. The user can then touch the subject's throat with a physically separate collection receptacle 1203 and touch the subject's throat with the collection member 1204 to collect the subject's throat specimen.

In certain cases, the tongue depressor used in the throat specimen collection devices described herein is popsicle shaped or a rectangular shaped. A rectangular shaped tongue depressor can have rounded corners. A tongue depressor can also be a semi-circle that matches with the contours of a subject's teeth. The shapes listed here are only exemplary and any suitable shape can be used that can fit in a subject's mouth and effectively restrict a subject's tongue without causing unnecessary discomfort.

A tongue depressor can be made of any suitable material, for example, a cardboard paper, rubber, plastic, or silicone.

Because a tongue depressor touches a subject's tongue, the tongue depressor can be configured to avoid unpleasant taste/flavor and/or to provide a pleasant taste/flavor. For example, a tongue depressor can be flavored, for example, by incorporating flavored substances into the materials used to produce the tongue depressor. When a tongue depressor touches a subject's tongue, such flavored material can dissolve into the subject's saliva and provide pleasant taste to the subject. In particular, tongue depressors with pleasant taste would facilitate obtaining throat specimens from children as children may be distracted from the unpleasant experience of taking the throat specimen and instead enjoy the pleasant taste.

In some cases, a tongue depressor is marked to indicate the appropriate location of the tongue depressor in a subject's mouth to restrain the subject's tongue. An appropriate location of a tongue depressor is such that when a subject's tongue is restrained by placing the tongue depressor in the subject's mouth, the collection member can properly touch the throat and collect a throat specimen from the subject. A marking on a tongue depressor that shows appropriate location for positioning the tongue depressor in a subject's mouth is designed to avoid the situations where: 1) the collection receptacle does not reach the subject's throat thereby failing to obtain a throat specimen or 2) the collection receptacle is too close to the subject's throat thereby possibly causing injury and/or pain.

### Methods of Collecting Throat Specimens

Also provided herein are methods of collecting throat specimen using the throat specimen collection devices described herein.

Accordingly, certain embodiments provide a method comprising:
i) positioning a tongue depressor of a throat specimen collection device to restrain a subject's tongue, and
ii) collecting a throat specimen of the subject when the collection receptacle is in the collection position.

As discussed above, in some embodiments of the devices, when a tongue depressor is positioned to restrain a subject's tongue, the collection receptacle is in the collection position, in which the collection member touches the throat of the subject and collects the throat specimen from the subject. Thus, when using such devices, the methods comprise: i) positioning the tongue depressor of the device to restrain the subject's tongue, and ii) collecting the throat specimen of the subject when the collection receptacle is in the collection position.

Also, in some embodiments of the devices, when a tongue depressor is positioned in a subject's mouth to restrain the subject's tongue, the collection receptacle is in the introduction position, where the collection member does not touch the throat of a subject. The collection receptacle in the introduction position can be moved into the collection position, where the collection member touches the throat of a subject and collects the throat specimen from the subject. Thus, when using such devices, the methods disclosed herein comprise: i) positioning the tongue depressor of the device to restrain a subject's tongue, and ii) moving the collection receptacle from the introduction position to the collection position thereby collecting the throat specimen of the subject.

Moreover, in some cases, collection receptacle is a physically separate unit but can be physically fitted into the tongue depressor. In such cases, when a tongue depressor is positioned in a subject's mouth to restrain the subject's tongue, the collection receptacle can be fitted into the tongue depressor at the introduction position, where the collection member does not touch the throat of a subject. The collection receptacle in the introduction position can be moved into the collection position, where the collection member touches the throat of a subject and collects the throat specimen from the subject. Thus, when using such devices, the methods disclosed herein comprise: i) positioning the tongue depressor of a throat specimen collection device to restrain a subject's tongue, ii) introducing the collection receptacle into the introduction position, and ii) moving the collection receptacle from the introduction position into the collection position thereby collecting the throat specimen of the subject.

In some cases, when a collection receptacle is a physically separate but can be physically fitted into a tongue depressor, the collection receptacle can be physically fitted into the tongue depressor before placing the tongue depressor in a subject's mouth. In such cases, the methods disclosed herein comprise: i) physically fitting the collection receptacle into the tongue depressor, ii) positioning the tongue depressor of the device to restrain the subject's tongue, and iii) collecting the throat specimen of the subject.

The throat specimen can be collected if the collection receptacle fitted into a tongue depressor is in a position where, when the tongue depressor of the device is positioned in a subject's mouth to restrain the subject's tongue, the collection member touches the throat of the subject. Alternatively, when the tongue depressor of the device is positioned in a subject's mouth to restrain the subject's tongue and the collection member does not touch the throat of the subject, the collection receptacle can be moved from the introduction position to the collection position thereby collecting the throat specimen of the subject.

As described above, certain throat specimen collection devices of the disclosure comprise an illuminator element. Accordingly, in certain embodiments, the methods disclosed herein comprise turning on the illuminator element of the throat specimen collection devices to illuminate the inside of a subject's mouth.

### Utility

The throat specimen collection devices and methods of collecting throat specimen as disclosed herein can be typically used in medical testing and diagnostics. As appreciated by a person of ordinary skill in the art, a throat specimen from a subject can be tested for infections, such as bacterial or viral infections. A throat specimen from a subject can be tested for collecting cells from a subject for DNA based diagnostics. Cells collected from a subject can also be tested for the presence of neoplasm.

In some cases, a throat specimen from a subject can be tested for detecting an infection with a pathogen, for example, a virus, a bacterium, or a fungus.

A pathogen that can be tested in a throat specimen from a subject includes one or more of human immunodeficiency virus (HIV), Hepatitis C virus (HCV), Hepatitis B virus (HBV), Chlamydia trachomatis (CT), Neisseria gonorrhoeae (NG), Human papillomavirus (HPV), Cytomegalovirus (CMV), Epstein-Barr virus (EBV), Polyomavirus BK (BKV), Methicillin-resistant Staphylococcus aureus (MRSA), Clostridium difficile (C. Diff.), Vancomycin-resistant enterococci (VRE), adenovirus, Mycobacterium tuberculosis (TB), Varicella Zoster Virus (VZV), Herpes simplex virus (HSV), John Cunningham virus (JCV), enterovirus, Lymphogranuloma Venereum (LGV), viruses of a Respiratory Viral Panel (RVP), Human Herpesvirus 6 (HHV6 ), Trichomonas vaginalis, Mycoplasma genitalium, norovirus, zika virus, Respiratory syncytial virus (RSV), streptococcal pathogen, such as Streptococcus pyrogenes and Streptococcus agalactiae, SARS-COV-2, and influenza virus.

A throat specimen from a subject can also be tested for toxicological screening, such as for detecting drugs or other chemicals. A drug or compound tested in a throat specimen includes alcohol, amphetamines, barbiturates, benzodiazepines, cocaine, marijuana, methamphetamine, opioids, opiates, and phencyclidine.

For reasons of completeness, various aspects of the invention are set out in the following numbered clauses:
Clause 1. A throat specimen collection device comprising:
   i) a tongue depressor, and
   ii) a collection receptacle comprising an elongated member and a collection member,
   wherein, when the tongue depressor is positioned to depress a subject's tongue and the collection receptacle is in a collection position, the collection member touches the throat of the subject and collects the throat specimen from the subject.
Clause 2. The throat specimen collection device of clause 1, wherein the collection receptacle is fitted in the tongue depressor such that when the tongue depressor is placed in the subject's mouth to restrain the subject's tongue, the collection receptacle is in the throat specimen collection position.
Clause 3. The throat specimen collection device of clause 1 or 2, wherein the collection receptacle is movable between an introduction position and the collection position.
Clause 4. The throat specimen collection device of clause 3, wherein the collection receptacle in the introduction position is slidable through the tongue depressor into the collection position.
Clause 5. The throat specimen collection device of any one of clauses 1 to 4, wherein the collection receptacle comprises the longitudinal member having at one end the collection member.
Clause 6. The throat specimen collection device of any one of clauses 1 to 5, wherein the collection member is a cotton swab, brush head, rayon swab, polyester swab, foam swab, or flocked swab.
Clause 7. The throat specimen collection device of clause 6, wherein the brush head of the collection member is a nubby silicon brush head.
Clause 8. The throat specimen collection device of any one of clauses 1 to 7, wherein the tongue depressor is a longitudinal flat member.
Clause 9. The throat specimen collection device of any one of clauses 1 to 7, wherein the tongue depressor is cylindrical.
Clause 10. The throat specimen collection device of any one of clauses 1 to 7, wherein the tongue depressor is popsicle shaped.
Clause 11. The throat specimen collection device of any one of clauses 1 to 10, wherein the tongue depressor is made of a cardboard paper.
Clause 12. The throat specimen collection device of any one of clauses 1 to 10, wherein the tongue depressor is made of silicone.
Clause 13. The throat specimen collection device of any one of clauses 1 to 10, wherein the tongue depressor is made of plastic.
Clause 14. The throat specimen collection device of any one of clauses 1 to 13, wherein the tongue depressor is flavored.
Clause 15. The throat specimen collection device of any one of clauses 1 to 14, wherein the introduction position of the collection receptacle is towards the subject's lips when the tongue depressor is positioned to restrain the subject's tongue.
Clause 16. A method of collecting a throat specimen of a subject using a throat specimen collection device, comprising:
   i) positioning a tongue depressor of the throat specimen collection device to restrain a subject's tongue, and
   ii) collecting a throat specimen of the subject when the collection receptacle is in the collection position.
Clause 17. The method of clause 16, further comprising moving the collection receptacle from an introduction position to the collection position.
Clause 18. The method of clause 17, further comprising introducing the collection receptacle at the introduction position before moving the collection receptacle from the introduction position to the collection position.
Clause 19. The method of any one of clauses 16 to 18, further comprising collecting the collection member to obtain the throat specimen from the collection receptacle.
Clause 20. A throat specimen collection device comprising:
   i) a tongue depressor configured to restrain a subject's tongue, and
   ii) a collection receptacle holder,
   wherein the tongue depressor is in a slidable engagement with the collection receptacle holder.
Clause 21. The throat specimen collection device of clause 20, wherein the tongue depressor is a longitudinal and flat element used to restrain a subject's tongue.
Clause 22. The throat specimen collection device of clause 20 or 21, wherein the slidable engagement comprises an elongated body on the tongue depressor, the elongated body engaging with the collection receptacle holder via a corresponding elongated slot which fits over the elongated body such that the collection receptacle holder slides over the elongated body of the tongue depressor.
Clause 23. The throat specimen collection device of any one of clauses 20 to 22, further comprising a rotatable engagement between the tongue depressor and the collection receptacle holder.
Clause 24. The throat specimen collection device of any one of clauses 20 to 23, wherein a collection receptacle is physically fitted on the collection receptacle holder.
Clause 25. The throat specimen collection device of any one of clauses 20 to 23, wherein the collection receptacle is physically integral with the collection receptacle holder.
Clause 26. The throat specimen collection device of any one of clauses 20 to 25, further comprising an illuminator element.
Clause 27. A throat specimen collection device comprising: a collection receptacle holder and an illuminator element.
Clause 28. The throat specimen collection device of clause 27, further comprising a collection receptacle.
Clause 29. The throat specimen collection device of clause 28, wherein the collection receptacle is physically fitted on the collection receptacle holder.
Clause 30. The throat specimen collection device of clause 28, wherein the collection receptacle is physically integral with the collection receptacle holder.
Clause 31. The throat specimen collection device of any one of clauses 27 to 30, further comprising a tongue depressor.
Clause 32. The throat specimen collection device of clause 31, wherein the tongue depressor is physically fitted into the device via a mounting element.
Clause 33. The throat specimen collection device of clause 31, wherein the tongue depressor is physically integral to the throat specimen collection device.
Clause 34. The throat specimen collection device of any one of clauses 20 to 34, wherein the tongue depressor has one of the following shapes: popsicle shaped, a rectangular shaped, a rectangular shaped with rounded corners, and a semi-circle that matches with the contours of a subject's teeth.
35. A method of collecting a throat specimen of a subject using a throat specimen collection device, the method comprising:
   i) positioning a tongue depressor of the throat specimen collection device to restrain the subject's tongue,
   ii) collecting a throat specimen of the subject when the collection receptacle is in a collection position.
Clause 36. The method of clause 35, further comprising moving the collection receptacle from an introduction position to the collection position.
Clause 37. The method of clause 36, further comprising introducing the collection receptacle at the introduction position before moving the collection receptacle from the introduction position to the collection position.
Clause 38. The method of any one of clauses 35 to 37, further comprising collecting a collection member to obtain the throat specimen from the collection receptacle.
Clause 39. The method of any one of clauses 38 to 38, wherein the throat specimen collection device comprises an illuminator element and the method comprises turning on the illuminator element and positioning the throat specimen collection device in the subject's mouth to illuminate the subject's mouth.

Although the foregoing disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present disclosure, therefore, is not intended to be limited to the exemplary embodiments shown and described herein. Rather, the scope and spirit of present disclosure is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

We claim:

1. A throat specimen collection device comprising:
i) a tongue depressor, and
ii) a collection receptacle comprising an elongated member and a collection member,
wherein, when the tongue depressor is positioned to depress a subject's tongue and the collection receptacle is in a collection position, the collection member touches the throat of the subject and collects the throat specimen from the subject.

2. The throat specimen collection device of claim 1, wherein the collection receptacle is fitted in the tongue depressor such that when the tongue depressor is placed in the subject's mouth to restrain the subject's tongue, the collection receptacle is in the throat specimen collection position.

3. The throat specimen collection device of claim 1 or 2, wherein the collection receptacle is movable between an introduction position and the collection position.

4. The throat specimen collection device of claim 3, wherein the collection receptacle in the introduction position is slidable through the tongue depressor into the collection position.

5. The throat specimen collection device of any one of claims 1 to 4, wherein the collection receptacle comprises the longitudinal member having at one end the collection member.

6. The throat specimen collection device of any one of claims 1 to 5, wherein the collection member is a cotton swab, brush head, rayon swab, polyester swab, foam swab, or flocked swab.

7. The throat specimen collection device of claim 6, wherein the brush head of the collection member is a nubby silicon brush head.

8. The throat specimen collection device of any one of claims 1 to 7, wherein the tongue depressor is a longitudinal flat member.

9. The throat specimen collection device of any one of claims 1 to 7, wherein the tongue depressor is cylindrical.

10. The throat specimen collection device of any one of claims 1 to 7, wherein the tongue depressor is popsicle shaped.

11. The throat specimen collection device of any one of claims 1 to 10, wherein the tongue depressor is made of a cardboard paper.

12. The throat specimen collection device of any one of claims 1 to 10, wherein the tongue depressor is made of silicone.

13. The throat specimen collection device of any one of claims 1 to 10, wherein the tongue depressor is made of plastic.

14. The throat specimen collection device of any one of claims 1 to 13, wherein the tongue depressor is flavored.

15. The throat specimen collection device of any one of claims 1 to 14, wherein the introduction position of the collection receptacle is towards the subject's lips when the tongue depressor is positioned to restrain the subject's tongue.

16. A method of collecting a throat specimen of a subject using a throat specimen collection device, comprising:
i) positioning a tongue depressor of the throat specimen collection device to restrain a subject's tongue, and
ii) collecting a throat specimen of the subject when the collection receptacle is in the collection position.

17. The method of claim 16, further comprising moving the collection receptacle from an introduction position to the collection position.

18. The method of claim 17, further comprising introducing the collection receptacle at the introduction position before moving the collection receptacle from the introduction position to the collection position.

19. The method of any one of claims 16 to 18, further comprising collecting the collection member to obtain the throat specimen from the collection receptacle.

20. A throat specimen collection device comprising:
i) a tongue depressor configured to restrain a subject's tongue, and
ii) a collection receptacle holder,
wherein the tongue depressor is in a slidable engagement with the collection receptacle holder.

21. The throat specimen collection device of claim 20, wherein the tongue depressor is a longitudinal and flat element used to restrain a subject's tongue.

22. The throat specimen collection device of claim 20 or 21, wherein the slidable engagement comprises an elongated body on the tongue depressor, the elongated body engaging with the collection receptacle holder via a corresponding elongated slot which fits over the elongated body such that the collection receptacle holder slides over the elongated body of the tongue depressor.

23. The throat specimen collection device of any one of claims 20 to 22, further comprising a rotatable engagement between the tongue depressor and the collection receptacle holder.

24. The throat specimen collection device of any one of claims 20 to 23, wherein a collection receptacle is physically fitted on the collection receptacle holder.

25. The throat specimen collection device of any one of claims 20 to 23, wherein the collection receptacle is physically integral with the collection receptacle holder.

26. The throat specimen collection device of any one of claims 20 to 25, further comprising an illuminator element.

27. A throat specimen collection device comprising: a collection receptacle holder and an illuminator element.

28. The throat specimen collection device of claim 27, further comprising a collection receptacle.

29. The throat specimen collection device of claim 28, wherein the collection receptacle is physically fitted on the collection receptacle holder.

30. The throat specimen collection device of claim 28, wherein the collection receptacle is physically integral with the collection receptacle holder.

31. The throat specimen collection device of any one of claims 27 to 30, further comprising a tongue depressor.

32. The throat specimen collection device of claim 31, wherein the tongue depressor is physically fitted into the device via a mounting element.

33. The throat specimen collection device of claim 31, wherein the tongue depressor is physically integral to the throat specimen collection device.

34. The throat specimen collection device of any one of claims 20 to 34, wherein the tongue depressor has one of the following shapes: popsicle shaped, a rectangular shaped, a rectangular shaped with rounded corners, and a semi-circle that matches with the contours of a subject's teeth.

35. A method of collecting a throat specimen of a subject using a throat specimen collection device, the method comprising:
i) positioning a tongue depressor of the throat specimen collection device to restrain the subject's tongue,
ii) collecting a throat specimen of the subject when the collection receptacle is in a collection position.

36. The method of claim 35, further comprising moving the collection receptacle from an introduction position to the collection position.

37. The method of claim 36, further comprising introducing the collection receptacle at the introduction position before moving the collection receptacle from the introduction position to the collection position.

38. The method of any one of claims 35 to 37, further comprising collecting a collection member to obtain the throat specimen from the collection receptacle.

39. The method of any one of claims 38 to 38, wherein the throat specimen collection device comprises an illuminator element and the method comprises turning on the illuminator element and positioning the throat specimen collection device in the subject's mouth to illuminate the subject's mouth.
